(19) United States
(12) Patent Application Publication
(10) Pub. No.: US 2021/0205170 A1
(43) Pub. Date: Jul. 8, 2021

(54) LASER ACUPUNCTURE TREATMENT APPARATUS

(71) Applicants: BOE TECHNOLOGY GROUP CO., LTD., Beijing (CN); HEFEI BOE OPTOELECTRONICS TECHNOLOGY CO., LTD., Anhui (CN)

(72) Inventor: Yangkun JING, Beijing (CN)

(21) Appl. No.: 16/074,154

(22) PCT Filed: Feb. 11, 2018
(86) PCT No.: PCT/CN2018/076317

§ 371 Date: Jul. 31, 2018

(30) Foreign Application Priority Data
May 12, 2017 (CN) 201710334973.2

(51) Int. Cl. A61H 39/08

(52) U.S. Cl. CPC A61H 39/086; A61H 2039/005

(57) ABSTRACT

A laser acupuncture treatment apparatus includes: a laser device; a collimating mirror disposed at a light exiting end of the laser device and configured to convert laser emitted from the laser device into a parallel laser beam; a laser polarizer disposed at a light exiting end of the collimating mirror and configured to convert the parallel laser beam into a first linearly polarized laser and a second linearly polarized laser, a polarization direction of the first linearly polarized laser being different from a polarization direction of the second linearly polarized laser; and a laser combiner disposed at a light exiting end of the laser polarizer and configured to combine the first linearly polarized laser and the second linearly polarized laser.

### CROSS REFERENCE

The present application is based on International Application No. PCT/CN2018/076317, filed on Feb. 11, 2018, which is based upon and claims priority to Chinese Patent Application No. 201710334973.2, entitled “Laser Acupuncture Treatment Apparatus”, filed on May 12, 2017, the entire contents of which are incorporated herein by reference.

### TECHNICAL FIELD

The present disclosure relates to the field of laser device technology, and more particularly, to a laser acupuncture treatment apparatus.

### BACKGROUND

Laser acupuncture is a new type of acupuncture method that irradiates little laser beam to an acupuncture point to treat a disease. Laser biological effects generally include thermal effects, light pressure effects, photochemical reactions, electromagnetic effects, and stimulating effects. Low-energy laser mainly has functions of anti-inflammation and promotion of the growth of epithelial cells. Compared with traditional acupuncture methods, the feature of the laser acupuncture is that there is no pain caused by acupuncture, no damage to the tissue, no sticking of the needle, no needle breakage or no needle infection. The laser acupuncture not only has similar acupuncture effect, but also has a series of biological effects caused by the laser.

The use of polarized laser for acupuncture in the laser acupuncture has obvious advantages, which is determined by the basic principle of polarized laser acupuncture: left-handed and right-handed molecules have different effects on polarized light, wherein one of them rotates the polarized light to the left, the other rotates the polarized light to the right. The basic substances for life are nucleic acids and proteins. Biomolecules have symmetry breaking phenomenon. For example, amino acids in a living body are the left-handed molecules, and ribose and deoxyribose molecules that make up the nucleic acid are all right-handed molecules. Basic biological molecules are both chiral and optically active. Through asymmetric photochemical synthesis and asymmetric photolysis, polarization properties of light become a natural motive force for the basic biological molecules to produce chirality and for high organization and ordering of organisms and become a motive force for creating life.

Therefore, in addition to physiotherapy effect of ordinary laser acupuncture, high coherence and polarization characteristics of the laser have a unique regulating effect on the highly organized and ordered human body. Many laser acupuncture researchers believe that the high coherence of the laser and the polarization characteristics of the beam are the keys to the unique physiotherapy effect of the laser acupuncture on the human body. Experiments have shown that the polarization characteristics of the laser have a unique role in acupuncture. How to obtain combined polarized light for laser acupuncture has become an urgent problem to be solved.

It should be noted that, information disclosed in the above background portion is provided only for better understanding of the background of the present disclosure, and thus it may contain information that does not form the prior art known by those ordinary skilled in the art.

### SUMMARY

In view of the above problems in the prior art, an object of the present disclosure is to provide a laser acupuncture treatment apparatus, which can obtain crisscross polarized laser for acupuncture and achieve a new acupuncture effect.

The present disclosure provides a laser acupuncture treatment apparatus, including: a laser device configured to emit laser; a collimating mirror disposed at a light exiting end of the laser device and configured to convert the laser emitted from the laser device into a parallel laser beam; a laser polarizer disposed at a light exiting end of the collimating mirror and configured to convert the parallel laser beam into a first linearly polarized laser and a second linearly polarized laser, a polarization direction of the first linearly polarized laser being different from a polarization direction of the second linearly polarized laser; and a laser combiner disposed at a light exiting end of the laser polarizer and configured to combine the first linearly polarized laser and the second linearly polarized laser to be outputted to a laser acupuncture needle.

The laser polarizer includes: a first laser polarizer configured to generate the first linearly polarized laser; and a second laser polarizer disposed side by side with the first laser polarizer and configured to generate the second linearly polarized laser.

The first laser polarizer and the second laser polarizer are slit structures.

The laser device is a He—Ne laser device.

The polarization direction of the first linearly polarized laser and the polarization direction of the second linearly polarized laser are perpendicular to each other.

The laser polarizer includes: a first rotatable polaroid disposed at the light exiting end of the collimating mirror and configured to convert the parallel laser beam into an elliptically polarized laser beam; a birefringent element disposed at a light exiting end of the first rotatable polaroid, and configured to perform birefringence of the elliptically polarized laser beam to obtain a first laser beam and a second laser beam; and a second rotatable polaroid disposed at a light exiting end of the birefringent element, and configured to rotatably polarize the first laser beam and the second laser beam to obtain the first linearly polarized laser and the second linearly polarized laser having different polarization directions.

The birefringent element includes calcite or barium titanate crystal.

The laser combiner is a focusing mirror.

The laser acupuncture treatment apparatus further includes: a laser acupuncture control module configured to control an intensity of the laser device and a position of laser acupuncture; and at least one image sensor connected to the laser acupuncture control module and configured to determine a position of an acupuncture point to be acupunctured.

The laser acupuncture treatment apparatus further includes: a laser power monitor connected to the collimating mirror and the laser polarizer, and configured to monitor a power of the laser outputted from the collimating mirror and a output power of polarized laser of a secondary polarization axis of the laser polarizer; and a laser power regulator connected to the laser device, and configured to compensate for an output power of polarized laser of a primary polarization axis of the laser polarizer based on the power of the laser outputted from the collimating mirror and the output power of the polarized laser of the secondary polarization axis of the laser polarizer.

The laser acupuncture treatment apparatus further includes an infrared temperature sensor connected to the laser combiner, and configured to detect a temperature of the laser emitted from the laser combiner.

The laser acupuncture treatment apparatus further includes: an optical path control module connected to the laser acupuncture control module, and configured to control a size of a light spot generated by the laser polarizer.

### BRIEF DESCRIPTION OF THE DRAWINGS

FIG. 1 is a structural schematic diagram of a laser acupuncture treatment apparatus according to an embodiment of the present disclosure.

FIG. 2 is a structural schematic diagram of a laser acupuncture treatment apparatus according to another embodiment of the present disclosure.

FIG. 3 is a structural schematic diagram of a laser acupuncture treatment apparatus according to still another embodiment of the present disclosure.

FIG. 4 is a perspective view of the laser acupuncture treatment apparatus shown in FIG. 3.

FIG. 5 is a structural schematic diagram of a laser acupuncture treatment apparatus according to a fourth embodiment of the present disclosure.

### DETAILED DESCRIPTION

The present disclosure will be further described in detail below in conjunction with the accompanying drawings and embodiments. It can be understood that specific embodiments described herein are merely illustrative of the present disclosure and are not restrictive of the present disclosure. In addition, it is also noted that, for the convenience of description, only part of not all of the structures related to the present disclosure are illustrated in the drawings.

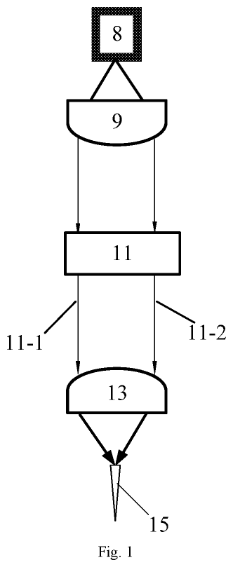

An embodiment of the present disclosure provides a laser acupuncture treatment apparatus. FIG. 1 is a structural schematic diagram of a laser acupuncture treatment apparatus according to an embodiment. As shown in FIG. 1, a laser acupuncture treatment apparatus according to an embodiment of the present disclosure includes a laser device 8, a collimating mirror 9, a laser polarizer 11, and a laser combiner 13. Although the laser device 8 can emit laser having polarization characteristics, the polarization direction of the emitted laser is not a constant direction, thus it is difficult to achieve an excellent acupuncture effect in terms of laser acupuncture. Moreover, the laser emitted from the laser device 8 is fan-shaped and divergent, and needs to be converted into a parallel laser beam. The laser device 8 is, for example, a He—Ne laser device, an infrared laser device or an ultraviolet laser device.

The collimating mirror 9 is disposed at a light exiting end of the laser device 8, and the laser emitted from the laser device 8 passes through the collimating mirror 9 and is converted into a parallel laser beam by the collimating mirror 9. The collimating mirror 9 is, for example, a plano-convex collimating mirror. The laser device 8 can be packaged in a housing made of a metallic material, a light guiding material or a reflective material such that the laser emitted from the laser device 8 is completely emitted toward the collimating mirror 9.

The laser polarizer 11 is disposed at a light exiting end of the collimating mirror 9, and the parallel laser beam emitted from the collimating mirror 9 passes through the laser polarizer 11 to obtain a first linearly polarized laser 11-1 and a second linearly polarized laser 11-2. A polarization direction of the first linearly polarized laser 11-1 and a polarization direction of the second linearly polarized laser 11-2 are different, and in one embodiment, are perpendicular to each other. The first linearly polarized laser 11-1 and the second linearly polarized laser 11-2 pass through the laser combiner 13 disposed at a light exiting end of the laser polarizer, and the laser combiner 13 is a focusing mirror, such as a plano-convex focusing mirror. The polarization direction of the first linearly polarized laser 11-1 is different from the polarization direction of the second linearly polarized laser 11-2, thus the effect of the present disclosure can be achieved, but if the polarization direction of the first linearly polarized laser 11-1 and the polarization direction of the second linearly polarized laser 11-2 are perpendicular to each other, due to the characteristics of biomolecules in a living body, the combined polarized laser may achieve better effects of acupuncture treatment and acupoint stimulation of a human body.

The laser combiner 13 combines the first linearly polarized laser 11-1 and the second linearly polarized laser 11-2 to obtain a combined crisscross polarized laser and form an energy wave having a combined polarization state, which doubles the acupoint effect, and makes stimulation and penetration of an acupuncture point stronger. The laser device 8, the collimating mirror 9, the laser polarizer 11 and the laser combiner 13 in this embodiment can be packaged together in a casing such that the crisscross polarized laser obtained after the combining is emitted from the casing to be outputted to a laser acupuncture needle 15. The crisscross polarized laser is used for acupuncture via the laser acupuncture needle 15 to improve the stimulation effect of the acupuncture point.

In the laser acupuncture treatment apparatus according to an embodiment of the present disclosure, the ordinary laser emitted from the laser device 8 is converted by the laser polarizer 11 into two linearly polarized laser which have different polarization directions, and in one embodiment, are perpendicular to each other, and the combined crisscross polarized laser can improve the effect of laser acupuncture, thus the laser acupuncture treatment apparatus can have a simple structure.

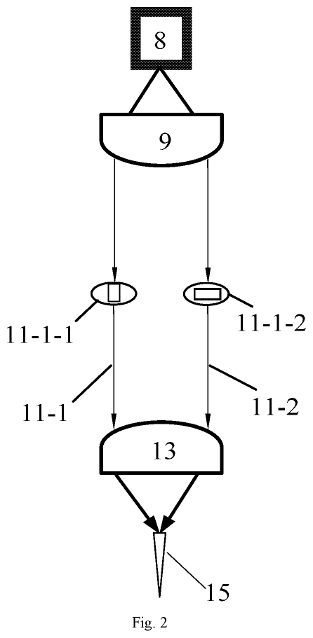

Another embodiment of the present disclosure provides a laser acupuncture treatment apparatus, which is a specific implementation form of the laser acupuncture treatment apparatus in the first embodiment. FIG. 2 is a structural schematic diagram of a laser acupuncture treatment apparatus according to another embodiment of the present disclosure. As shown in FIG. 2, the laser acupuncture treatment apparatus according to another embodiment of the present disclosure includes a laser device 8, a collimating mirror 9, a first laser polarizer 11-1-1, a second laser polarizer 11-1-2, and a laser combiner 13.

Although the laser device 8 can emit laser having polarization characteristics, the polarization direction of the emitted laser is not a constant direction, thus it is difficult to achieve an excellent acupuncture effect in terms of laser acupuncture. The collimating mirror 9 is disposed at a light exiting end of the laser device 8, and the laser emitted from the laser device 8 passes through the collimating mirror 9 and is converted into a parallel laser beam by the collimating mirror 9. The first laser polarizer 11-1-1 and the second laser polarizer 11-1-2 are disposed side by side at a light exiting end of the collimating mirror 9. The parallel laser beam emitted from the collimating mirror 9 passes through the first laser polarizer 11-1-1 to obtain a first linearly polarized laser 11-1; meanwhile, the parallel laser beam emitted from the collimating mirror 9 passes through the second laser polarizer 11-1-12 to obtain a second linearly polarized laser 11-2. A polarization direction of the first laser polarizer 11-1-1 and a polarization direction of the second laser polarizer 11-1-2 are different, and in one embodiment, are perpendicular to each other, such that polarization directions of the obtained first linearly polarized laser 11-1 and second linearly polarized laser 11-2 are different, and in one embodiment, are perpendicular to each other. Here, the first laser polarizer 11-1-1 and the second laser polarizer 11-1-2 can be configured to have slit structures. The laser passes through slits of the first laser polarizer 11-1-1 and the second laser polarizer 11-1-2, respectively to emit polarized laser which have different polarization directions, and in one embodiment, are perpendicular to each other.

The first linearly polarized laser 11-1 and the second linearly polarized laser 11-2 pass through the laser combiner 13 disposed at light exiting ends of the first laser polarizer 11-1-1 and the second laser polarizer 11-1-2, and the laser combiner 13 is a focusing mirror.

The laser combiner 13 combines the first linearly polarized laser 11-1 and the second linearly polarized laser 11-2 to obtain a combined crisscross polarized laser and form an energy wave having a combined polarization state, which doubles the acupoint effect, and makes stimulation and penetration of an acupuncture point stronger. The crisscross polarized laser is used for acupuncture via a laser acupuncture needle 15 to improve the stimulation effect of the acupuncture point.

In another embodiment of the present disclosure, the laser polarizer 11 is composed of the first laser polarizer 11-1-1 and the second laser polarizer 11-1-2, which are disposed side by side and have two different directions perpendicular to each other. The first laser polarizer 11-1-1 and the second laser polarizer 11-1-2 may convert the ordinary laser emitted from the laser device 8 into the first linearly polarized laser 11-1 and the second linearly polarized laser 11-2 which are different in polarization direction, and in one embodiment, are perpendicular to each other, to be combined to obtain a crisscross polarized laser, in this way, the effect of laser acupuncture can be improved, and the laser acupuncture treatment apparatus can have a simple structure.

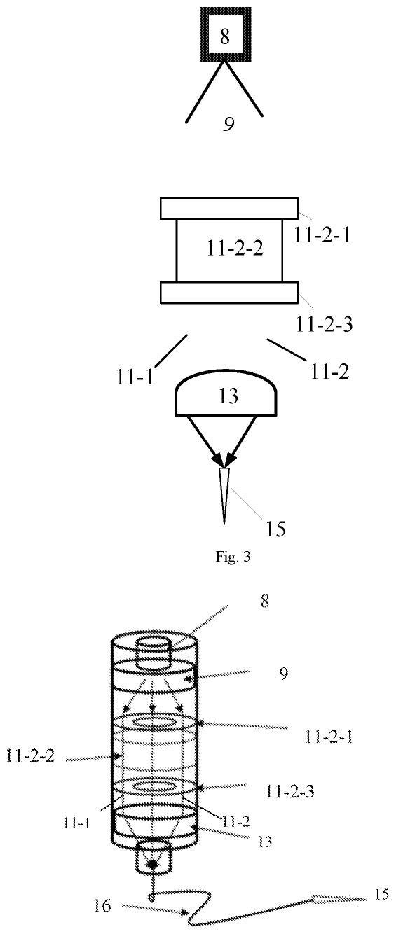

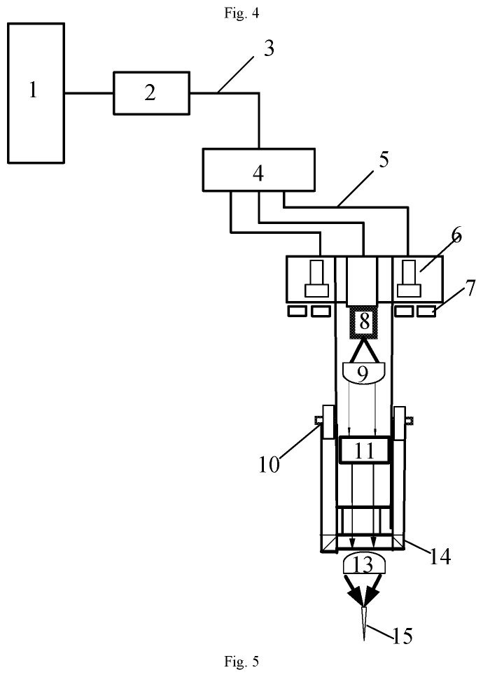

Another embodiment of the present disclosure provides a laser acupuncture treatment apparatus, which is another specific implementation form of the laser acupuncture treatment apparatus in the first embodiment. FIG. 3 is a structural schematic diagram of a laser acupuncture treatment apparatus according to still another embodiment of the present disclosure. FIG. 4 is a perspective view of the laser acupuncture treatment apparatus shown in FIG. 3.

As shown in FIGS. 3 and 4, the laser acupuncture treatment apparatus according to the present embodiment includes a laser device 8, a collimating mirror 9, a first rotatable polaroid 11-2-1, a birefringent element 11-2-2, a second rotatable polaroid 11-2-3, and a laser combiner 13.

Although the laser device 8 can emit laser having polarization characteristics, the polarization direction of the emitted laser is not a constant direction, thus it is difficult to achieve an excellent acupuncture effect in terms of laser acupuncture. The collimating mirror 9 is disposed at a light exiting end of the laser device 8, and the laser emitted from the laser device 8 passes through the collimating mirror 9 and is converted into a parallel laser beam by the collimating mirror 9, and the first rotatable polaroid 11-2-1 is disposed at a light exiting end of the collimating mirror 9, the first rotatable polaroid 11-2-1 converts the parallel laser beam into an elliptically polarized laser beam.

The birefringent element 11-2-2 is disposed at a light exiting end of the first rotatable polaroid 11-2-1, and carries out birefringence of the elliptically polarized laser beam to obtain a first laser beam and a second laser beam. The birefringent element 11-2-2 can be calcite or barium carbonate crystal. The calcite does not need to be energized, but an electric field needs to be added for the barium carbonate crystal. The advantage of the calcite is that its birefringence is not controllable, but birefringence of the barium carbonate crystal is controllable.

The principle in which the birefringent element 11-2-2 carries out birefringence of the elliptically polarized laser beam to obtain the first laser beam and the second laser beam will be described below.

When light is incident on an optical birefringence substance, a propagation speed of the light is different in an xy amplitude direction.

(speed of light in a medium)=(speed of light in vacuum)/(refractive index)

Light vector can be expressed as follows:

(light vector)=A cos[2πγ(t−x/v)+φ]

Where A represents amplitude; γ represents an optical frequency; v represents the speed of light in the medium; φ represents an initial phase. A vector of light in x and y directions can be decomposed into: E=E x+E y, and when the light is understood as a wave, the following can be derived:

Ex=A cos[2πγ(t−x/vx)+φ]

Ey=A cos[2πγ(t−x/vy)+φ]

Therefore, the birefringent element 11-2-2 performs birefringence of the elliptically polarized laser beam to obtain the first laser beam and the second laser beam having different vectors.

The second rotatable polaroid 11-2-3 is disposed at a light exiting end of the birefringent element 11-2-2, and rotationally polarizes the first laser beam and the second laser beam to obtain a first linearly polarized laser 11-1 and a second linearly polarized laser 11-2 having different polarization directions, and in one embedment, the polarization directions of the first linearly polarized laser 11-1 and the second linearly polarized laser 11-2 are perpendicular to each other.

The parallel laser beam emitted from the collimating mirror 9 passes through the first rotatable polaroid 11-2-1, the birefringent element 11-2-2, and the second rotatable polaroid 11-2-3 to obtain the first linearly polarized laser 11-1 and the second linearly polarized laser 11-2, wherein the polarization direction of the first linearly polarized laser 11-1 and the polarization direction of the second linearly polarized laser 11-2 are different, and in one embodiment, are perpendicular to each other.

The first linearly polarized laser 11-1 and the second linearly polarized laser 11-2 pass through the laser combiner 13 disposed at a light exiting end of the second rotatable polaroid 11-2-3, and the laser combiner 13 is a focusing mirror.

The laser combiner 13 combines the first linearly polarized laser 11-1 and the second linearly polarized laser 11-2 to obtain a combined crisscross polarized laser and form an energy wave having a combined polarization state, which doubles the acupoint effect, and makes stimulation and penetration of an acupuncture point stronger. The crisscross polarized laser is used for acupuncture via a laser acupuncture needle 15 to improve the stimulation effect of the acupuncture point.

In the present embodiment, a laser polarizer 11 is formed by sequentially disposing the first rotatable polaroid 11-2-1, the birefringent element 11-2-2, and the second rotatable polaroid 11-2-3 in a traveling direction of the laser, and may convert the ordinary laser emitted from the laser device 8 into the first linearly polarized laser 11-1 and the second linearly polarized laser 11-2 having different polarization directions, and in one embodiment, are perpendicular to each other, to be combined to obtain the crisscross polarized laser, which can improve the effect of laser acupuncture.

A yet another embodiment of the present disclosure provides a laser acupuncture treatment apparatus including the structure members of the laser acupuncture treatment apparatus in the first embodiment to the third embodiment, and further including other members for controlling an intensity of laser and an acupuncture position. FIG. 5 is a structural schematic diagram of a laser acupuncture treatment apparatus according to yet another embodiment of the present disclosure.

As shown in FIG. 5, the laser acupuncture treatment apparatus according to the present embodiment includes a PLC controller 1 configured to process height information of a detected acupuncture point, and controlling an intensity of laser and an acupuncture position; a signal converter 2 connected to the PLC controller 1, and configured to convert a signal generated by the PLC controller 1; a signal line 3; an integrated processing module 4 connected to the signal converter 2 through the signal line 3, and the signal converted by the signal converter 2 being transmitted to the integrated processing module 4 via the signal line 3. The integration of these members can be called a laser acupuncture control module, i.e., a control host, in the laser acupuncture treatment apparatus. The control host includes a power supply circuit, a pulse generation and modulation circuit, a laser control circuit, a drive circuit, and an infrared control and feedback circuit; the pulse generation and modulation circuit generates a signal to directly control an output of an infrared ray tube in a laser device, and generates a signal to control an output of the laser device by the laser control circuit and the drive circuit.

The laser acupuncture treatment apparatus of the present embodiment further includes an image sensor 6 and a diaphragm 7 for determining a position of an acupuncture point to be acupunctured. The present embodiment includes two image sensors, wherein adjustment of the position can be performed by the image sensors, and positioning of a corresponding acupuncture point is performed by using a meridian-collateral diagram of a human body; and a laser acupuncture treatment apparatus. The image sensor 6 and the laser acupuncture treatment apparatus are connected to the integrated processing module 4 via a signal line 5, respectively. The image sensor 6 and the laser acupuncture treatment apparatus are connected to the aforementioned laser acupuncture control module through the signal line, and the aforementioned laser acupuncture control module controls the laser acupuncture treatment apparatus based on a signal received from the image sensor 6. The image sensor can be a camera or a video camera.

In addition, the laser acupuncture treatment apparatus of the present embodiment further includes an optical path control module 10 and an infrared temperature sensing module 14 connected to the laser acupuncture control module and the laser acupuncture treatment apparatus, wherein the optical path control module 10 is configured to control a size of a light spot of the crisscross laser outputted by the laser acupuncture treatment apparatus, the optical path control module can be an optical path controller or other devices capable of implementing optical path control; and the infrared temperature sensing module 14 is configured to sense and control a temperature of the crisscross laser outputted from the laser acupuncture treatment apparatus, and the infrared temperature sensing module can be an infrared temperature sensor.

As described in the first embodiment, the laser acupuncture treatment apparatus includes a laser device 8, a collimating mirror 9, a laser polarizer 11, and a laser combiner 13.

The laser acupuncture treatment apparatus of the present embodiment further includes a laser power monitor (not shown) connected to the collimating mirror and the laser polarizer and configured to monitor a power of laser outputted from the collimating mirror and an output power of a polarized laser of a secondary polarization axis of the laser polarizer; and a laser power regulator (not shown) connected to the laser device and configured to compensate for an output power of a polarized laser of a primary polarization axis of the laser polarizer according to the power of the laser outputted from the collimating mirror and the output power of the polarized laser of the secondary polarization axis of the laser polarizer.

The power of the laser outputted from the collimating mirror 9 is proportional to a total power outputted from the laser device, and the laser power monitor monitors the power of laser outputted from the collimating mirror 9 to obtain the total power outputted from the laser device; and then the laser power monitor monitors the output power of the polarized laser of the secondary polarization axis of the laser polarizer 11, so as to obtain the output power of the polarized laser of the primary polarization axis of the laser polarizer by subtracting the output power of the polarized laser of the secondary polarization axis from the total power; finally, the laser power regulator regulates a drive current of the laser device 8 to change a power of a pump laser to compensate for fluctuation of the output power of the polarized laser of the primary polarization axis caused by changes in a polarization state of the laser device, and obtain an output of the polarized laser of the primary polarization axis having a stable power.

Steps of carrying out acupuncture treatment by using the laser acupuncture treatment apparatus of the present embodiment are as follows: the laser acupuncture treatment apparatus receiving a coordinate point of an acupuncture point transmitted by a feature recognition system of a meridian-collateral diagram of a human body in a storage device; the laser acupuncture needle 15 moving based on the coordinate point, and the image sensor 6 stopping above the detected acupuncture point, focusing on the acupuncture point, and taking a photo, and then transmitting the photographed information to the laser acupuncture control module. The laser acupuncture control module identifies a maximum point of gray difference of the acupuncture point as a center of the acupuncture point by performing differential process of an image. By checking and judging a shape of the acupuncture point, the laser acupuncture control module regulates and controls the laser device in the laser acupuncture treatment apparatus to emit a laser of an appropriate intensity. The laser passes through the laser polarizer, which adjusts and controls the intensity and a polarization direction of the polarized light to obtain linearly polarized laser which have different polarization directions, and in one embodiment, are perpendicular to each other, and then the linearly polarized laser having different polarization directions are combined to be outputted to the laser acupuncture needle 15 to irradiate the acupuncture point, and after the acupuncture point absorbs light, surface stimulation is generated, in this way, a brand new acupuncture effect is achieved. During this procedure, the optical path control module connected to the laser acupuncture control module can be used to control the size of the light spot generated by the laser polarizer, and a temperature outputted to the laser acupuncture needle 15 is sensed by the infrared temperature sensor connected to the laser combiner.

According to the laser acupuncture treatment apparatus of the present embodiment, the polarized light is concentrated to an acupuncture point by the laser polarizer and the laser combiner, and the two linear polarizers in the second embodiment or the rotatable polaroid and the birefringent element in the third embodiment are utilized to combine the polarized light, and the polarized laser with different directions can be synchronously controlled to be combined to form an energy wave having a combined polarization state, which doubles the acupoint effect, and makes stimulation and penetration of the acupuncture point stronger. The image sensor of the laser acupuncture treatment apparatus can adjust the position. The laser acupuncture control module can rotatably regulate and control the intensity and combination direction of the polarized light and output the polarized laser of the primary polarization axis having stable power, thereby achieving a brand new acupuncture effect.

It is noted that the foregoing contents are only the preferred embodiments of the present disclosure and the employed technical principles. It should be understood by those skilled in the art that the present disclosure is not limited to the specific embodiments described herein, and that various obvious changes, readjustments and substitutions may be made by those skilled in the art without departing from the protection scope of the present disclosure. Therefore, although the present disclosure has been described in detail with reference to the above embodiments, the present disclosure is not limited to only the above embodiments, and more other equivalent embodiments can be included without departing from the concept of the present disclosure. The scope of the present disclosure is determined by the appended claims.

1. A laser acupuncture treatment apparatus, comprising:
a laser device, configured to emit laser;
a collimating mirror, disposed at a light exiting end of the laser device and configured to convert the laser emitted from the laser device into a parallel laser beam;
a laser polarizer, disposed at a light exiting end of the collimating mirror and configured to convert the parallel laser beam into a first linearly polarized laser and a second linearly polarized laser, a polarization direction of the first linearly polarized laser being different from a polarization direction of the second linearly polarized laser; and
a laser combiner, disposed at a light exiting end of the laser polarizer and configured to combine the first linearly polarized laser and the second linearly polarized laser to be outputted to a laser acupuncture needle.

2. The laser acupuncture treatment apparatus according to claim 1, wherein the polarization direction of the first linearly polarized laser and the polarization direction of the second linearly polarized laser are perpendicular to each other.

3. The laser acupuncture treatment apparatus according to claim 1, wherein the laser polarizer comprises:
a first laser polarizer, configured to convert the parallel laser beam into the first linearly polarized laser; and
a second laser polarizer, disposed side by side with the first laser polarizer and configured to convert the parallel laser beam into the second linearly polarized laser.

4. The laser acupuncture treatment apparatus according to claim 3, wherein the first laser polarizer and the second laser polarizer are slit structures.

5. The laser acupuncture treatment apparatus according to claim 1, wherein the laser device is a He—Ne laser device.

6. The laser acupuncture treatment apparatus according to claim 1, wherein the laser polarizer comprises:
a first rotatable polaroid, disposed at the light exiting end of the collimating mirror and configured to convert the parallel laser beam into an elliptically polarized laser beam;
a birefringent element, disposed at a light exiting end of the first rotatable polaroid, and configured to perform birefringence of the elliptically polarized laser beam to obtain a first laser beam and a second laser beam; and
a second rotatable polaroid, disposed at a light exiting end of the birefringent element, and configured to rotatably polarize the first laser beam and the second laser beam to obtain the first linearly polarized laser and the second linearly polarized laser having different polarization directions.

7. The laser acupuncture treatment apparatus according to claim 6, wherein the polarization direction of the first linearly polarized laser and the polarization direction of the second linearly polarized laser are perpendicular to each other.

8. The laser acupuncture treatment apparatus according to claim 6, wherein the birefringent element comprises calcite or barium titanate crystal.

9. The laser acupuncture treatment apparatus according to claim 1, wherein the laser combiner is a focusing mirror.

10. The laser acupuncture treatment apparatus according to claim 1, further comprising:
a laser acupuncture control module, configured to control an intensity of the laser device and a position of laser acupuncture; and
at least one image sensor, connected to the laser acupuncture control module and configured to determine a position of an acupuncture point to be acupunctured.

11. The laser acupuncture treatment apparatus according to claim 10, further comprising:
a laser power monitor, connected to the collimating mirror and the laser polarizer, and configured to monitor a power of the laser outputted from the collimating mirror and an output power of polarized laser of a secondary polarization axis of the laser polarizer; and
a laser power regulator, connected to the laser device, and configured to compensate for an output power of polarized laser of a primary polarization axis of the laser polarizer based on the power of the laser outputted from the collimating mirror and the output power of the polarized laser of the secondary polarization axis of the laser polarizer.

12. The laser acupuncture treatment apparatus according to claim 10, further comprising:
an infrared temperature sensor, connected to the laser combiner, and configured to detect a temperature of the laser emitted from the laser combiner.

13. The laser acupuncture treatment apparatus according to claim 10, further comprising:
an optical path control module, connected to the laser acupuncture control module, and configured to control a size of a light spot generated by the laser polarizer.

14. The laser acupuncture treatment apparatus according to claim 2, wherein the laser polarizer comprises:
a first rotatable polaroid, disposed at the light exiting end of the collimating mirror and configured to convert the parallel laser beam into an elliptically polarized laser beam;
a birefringent element, disposed at a light exiting end of the first rotatable polaroid, and configured to perform birefringence of the elliptically polarized laser beam to obtain a first laser beam and a second laser beam; and
a second rotatable polaroid, disposed at a light exiting end of the birefringent element, and configured to rotatably polarize the first laser beam and the second laser beam to obtain the first linearly polarized laser and the second linearly polarized laser having different polarization directions.

15. The laser acupuncture treatment apparatus according to claim 3, wherein the laser polarizer comprises:
a first rotatable polaroid, disposed at the light exiting end of the collimating mirror and configured to convert the parallel laser beam into an elliptically polarized laser beam;
a birefringent element, disposed at a light exiting end of the first rotatable polaroid, and configured to perform birefringence of the elliptically polarized laser beam to obtain a first laser beam and a second laser beam; and
a second rotatable polaroid, disposed at a light exiting end of the birefringent element, and configured to rotatably polarize the first laser beam and the second laser beam to obtain the first linearly polarized laser and the second linearly polarized laser having different polarization directions.

16. The laser acupuncture treatment apparatus according to claim 4, wherein the laser polarizer comprises:
a first rotatable polaroid, disposed at the light exiting end of the collimating mirror and configured to convert the parallel laser beam into an elliptically polarized laser beam;
a birefringent element, disposed at a light exiting end of the first rotatable polaroid, and configured to perform birefringence of the elliptically polarized laser beam to obtain a first laser beam and a second laser beam; and
a second rotatable polaroid, disposed at a light exiting end of the birefringent element, and configured to rotatably polarize the first laser beam and the second laser beam to obtain the first linearly polarized laser and the second linearly polarized laser having different polarization directions.

17. The laser acupuncture treatment apparatus according to claim 2, further comprising:
a laser acupuncture control module, configured to control an intensity of the laser device and a position of laser acupuncture; and
at least one image sensor, connected to the laser acupuncture control module and configured to determine a position of an acupuncture point to be acupunctured.

18. The laser acupuncture treatment apparatus according to claim 3, further comprising:
a laser acupuncture control module, configured to control an intensity of the laser device and a position of laser acupuncture; and
at least one image sensor, connected to the laser acupuncture control module and configured to determine a position of an acupuncture point to be acupunctured.

19. The laser acupuncture treatment apparatus according to claim 4, further comprising:
a laser acupuncture control module, configured to control an intensity of the laser device and a position of laser acupuncture; and
at least one image sensor, connected to the laser acupuncture control module and configured to determine a position of an acupuncture point to be acupunctured.

20. The laser acupuncture treatment apparatus according to claim 5, further comprising:
a laser acupuncture control module, configured to control an intensity of the laser device and a position of laser acupuncture; and
at least one image sensor, connected to the laser acupuncture control module and configured to determine a position of an acupuncture point to be acupunctured.
